(19) United States
(12) Patent Application Publication
(10) Pub. No.: US 2005/0019288 A1
(43) Pub. Date: Jan. 27, 2005

(54) Alcohol-based aerosol deodorant composition comprising at lease one zinc salicylate or a derivative thereof

(76) Inventor: Cyril Lemoine, Puiseux-en-France (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/857,975

(22) Filed: Jun. 2, 2004

Related U.S. Application Data
(60) Provisional application No. 60/519,260, filed on Nov. 13, 2003.

(30) Foreign Application Priority Data
Jun. 2, 2003 (FR) 03 06622

(51) Int. Cl. A61K 7/32; A61K 7/36

(52) U.S. Cl. 424/67

(57) ABSTRACT

Disclosed herein is a deodorant composition, characterized in that it contains, in a cosmetically acceptable vehicle: (a) at least one zinc salicylate or a derivative thereof, in free or hydrated form;

(b) at least one C₁-C₄ monoalcohol; and (c) at least one propellant.

Also disclosed herein is an aerosol device comprising a container comprising an aerosol composition comprising firstly of a fluid phase comprising at least one C₁-C₄ monoalcohol, at least zinc salicylate or a derivative thereof, in free or hydrated form, at least one propellant, and a means for distributing the aerosol composition. Further disclosed herein is a process for treating human body odor, and especially underarm odor, using this device.

This application claims benefit of U.S. Provisional Application No. 60/519,260, filed Nov. 13, 2003.

Disclosed herein is a deodorant composition, comprising, in a cosmetically acceptable vehicle:

(a) at least one zinc salicylate or a derivative thereof, in free or hydrated form;

(b) at least one C₁-C₄ monoalcohol; and

(c) at least one propellant.

Also disclosed herein is an aerosol device comprising a container comprising an aerosol composition comprising a fluid (or liquid) comprising at least one C₁-C₄ monoalcohol; at least one zinc salicylate or a derivative thereof, in free or hydrated form; and at least one propellant; and a means for distributing the aerosol composition.

Further disclosed herein is a process for treating human body odor, such as underarm odor, using this device.

Also disclosed herein is the use of zinc salicylate or a derivative thereof, in free or hydrated form, as a deodorant active agent in a composition packaged in aerosol form containing at least one C₁-C₄ monoalcohol in a cosmetically acceptable vehicle.

As used herein, the term “zinc salicylate derivative” means any compound corresponding to the structure: 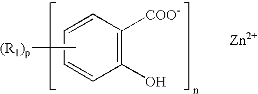
wherein n is 2 and p is 0, 1, 2 or 3 and R₁ is a substituent.

In the cosmetic field, it is well known to use, in topical application, deodorant products containing active substances, of antiperspirant type or of bactericidal type, to reduce or even eliminate underarm odors.

Antiperspirant substances have the effect of limiting the flow of sweat. They are generally comprised of aluminium salts, which, on the one hand, may be irritating to the skin, and, on the other hand, may reduce the flow of sweat by modifying the cutaneous physiology, both of which can be unsatisfactory.

Bactericidal substances may inhibit the growth of the cutaneous flora responsible for underarm odors.

Among the bactericidal products most commonly used is Triclosan (2,4,4′-trichloro-2′-hydroxydiphenyl ether), which has the drawback of substantially modifying the ecology of the cutaneous flora. 3,7,11-Trimethyldodeca-2,5,10-trienol (Farnesol) is also known, but has the drawback of being allergenic.

With the aim of obtaining long-term efficacy, novel products are sought that exert deodorant action, i.e., products that are capable of modifying, reducing and/or removing or preventing the development of body odor (this definition is given in. COSMETIC SCIENCE AND TECHNOLOGY SERIES Chapter 10-IIIc (Vol. 7. 1988)). In addition, products are sought that do not have the drawbacks of the active substances used in the prior art.

In this regard, certain water-soluble zinc salts have already been proposed as deodorant active agents in deodorant products, for instance zinc pyrrolidonecarboxylate (more commonly known as zinc pidolate), zinc sulphate, zinc chloride, zinc lactate, zinc gluconate and zinc phenolsulphonate. Such deodorant formulations have been described, for example, in documents WO 93/01793, EP 468 564, EP 024 176 and EP 768 080. However, such zinc-based odor absorbers are unsatisfactory due to the fact that most of them are difficult to formulate in alcohol-based aerosols, which nowadays represent the majority of the deodorant products on the market.

In the 1930s, as described in particular in the book DRUGS & COSMETIC INDUSTRY-MODERN COSMETICS 168 (1934), it was known that zinc salicylate could be formulated in deodorant formulations of paste type or in stick form in the necessary presence of other active agents for a sufficient deodorant action. It has more recently been proposed as a possible deodorant active agent in various supports: according to patent EP 318 206 in stick gels based on benzylidene sorbitol, an emollient of the diisopropyl adipate type, one or more polar solvents and a coupling agent; according to patents U.S. Pat. No. 5,254,332 and U.S. Pat. No. 5,302,381 in sticks containing a volatile silicone oil, a low-melting wax, a liquid water-insoluble emollient and in the presence of a deodorant active agent of the sodium bicarbonate type; according to patent application WO 02/13776 in aqueous gels based on fatty acid soaps.

After extensive research conducted in this matter, the present inventor has now found, surprisingly and unexpectedly, that, firstly, zinc salts of the salicylate type may have a bactericidal activity spectrum that is substantially less broad than that of Triclosan and thus may be friendlier towards the cutaneous flora, and, secondly, they can be formulated in a standard alcohol-based aerosol to obtain a formulation that may be stable on storage and, finally, they may have good deodorant efficacy.

This discovery forms the basis of the present invention.

The at least one zinc salicylate and derivatives thereof disclosed herein generally correspond to the following- structure: 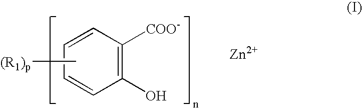
wherein n=2, p is 0, 1, 2 or 3; R₁ is chosen from linear and branched C₁-C₁₈ alkyl radicals (for example methyl, ethyl, n-propyl, isopropyl or n-butyl); linear and branched C₁-C₁₈ hydroxyalkyl radicals; halogen atoms (for example iodine, bromine or chlorine); C₂-C₁₈ acyl radicals (for example acetyl); radicals chosen from COR₂, OCOR₂ and CONHR₂ wherein R₂ is chosen from hydrogen atoms and linear and branched C₁-C₁₈ alkyl radicals.

In one embodiment, zinc salicylate itself (where p=0) may be chosen, for example in hydrated form, such as zinc salicylate trihydrate, for example the product sold under the name “Zinc Salicylate Trihydrate” by the company Bernardy Chimie.

In the compositions disclosed herein, the concentration of the at least one zinc salicylate or derivatives thereof ranges, for example, from 0.001% to 20%, further for example, from 6.01% to 15%, and even further for example, from 0.05% to 5% by weight relative to the total weight of the composition.

The C₁-C₄ monoalcohol(s) present in the compositions disclosed herein may be chosen from methanol, ethanol and isopropanol, or mixtures thereof. Ethanol, for example, may be chosen. The C₁-C₄ monoalcohol(s) are generally present at concentrations ranging from, for example, 15% to 80%, further for example from 30% to 70%, and even further for example from 40% to 70% by weight relative to the total weight of the composition.

The at least one propellant may be chosen from dimethyl ether; volatile hydrocarbons such as n-butane, propane, isopropane, n-butane, isobutane, pentane and isopentane, and mixtures thereof; volatile fluorohydrocarbons such as 1,1-difluoroethane and 1,1,1-trifluoro-2-fluoroethane. Carbon dioxide, nitrous oxide, nitrogen or compressed air may also be used as propellant.

The composition comprising at least one zinc salicylate or derivative(s) thereof and the at least one propellant may be in the same compartment or in different compartments in the aerosol container.

The concentration of the at least one propellant generally ranges, for example, from 5% to 90% by pressurized weight, and further for example, from 15% to 70% by weight relative to the total weight of the pressurized composition.

In addition to the at least one zinc salicylate, the composition disclosed herein may further comprise other additional deodorant active agents or antiperspirant active agents.

The additional deodorant active agents that may be mentioned, for example, are: zinc pyrrolidonecarboxylate (more commonly known as zinc pidolate), zinc sulphate, zinc chloride, zinc lactate, zinc gluconate and zinc phenolsulphonate, 2,4,4′-trichloro-2′-hydroxydiphenyl ether (Triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (Triclocarban) or 3,7,11-trimethyldodeca-2,5,10-trienol (Farnesol); quaternary ammonium salts, for instance cetyltrimethylammonium salts and cetylpyridinium salts; chlorhexidine and salts; diglyceryl monocaprate, diglyceryl monolaurate and glyceryl monolaurate; and pqlyhexamethylene biguanide salts.

The active agents can be chosen from, for example, 2,4,4′-trichloro-2′-hydroxydiphenyl ether (Triclosan); polyhexamethylene biguanide salts (known as polyaminopropyl biguanide salts), for example the product Cosmocil CQTM (Zeneca); and 3,7,11-trimethyldodeca-2,5,10-trienol (Farnesol).

The additional antiperspirant active agents that may be mentioned, for example, are aluminium chlorohydrate, aluminium chlorohydrex, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, aluminium sulphate, aluminium zirconium octachlorohydrate, aluminium zirconium pentachlorohydrate, aluminium zirconium tetrachlorohydrate, aluminium zirconium trichlorohydrate, and complexes of metals (for instance aluminium or zirconium) with an amino acid, as described in patent U.S. Pat. No. 3,792,068. Such complexes are generally known under the name ZAG (when the amino acid is glycine). The ZAG complexes usually have an Al/Zr ratio ranging from about 1.67 to 12.5 and a metal/Cl ratio ranging from about 0.73 to 1.93.

An amino acid that can be used, for example, to prepare the ZAG complexes, is glycine. Among these products, mention may be made of aluminium zirconium octachlorohydrex GLY, aluminium zirconium pentachlorohydrex GLY, aluminium zirconium tetrachlorohydrate GLY and aluminium zirconium trichlorohydrate GLY.

These additional deodorant or antiperspirant active agents may be present in the composition disclosed herein in a concentration ranging, for example, from 0.001 % to 20%, and further for example, from 0.1 % to 10% by weight relative to the total composition.

In one embodiment, the alcohol-based aerosol deodorant composition does not contain any additional deodorant or antiperspirant active agent such as those mentioned above.

The cosmetically acceptable vehicle, which is alcohol-based, may comprise water and/or at least one additional organic solvents, for example, propylene glycol and dipropylene glycol, or ethers thereof.

The cosmetic composition disclosed herein may also be formulated as a water-in-oil or oil-in-water emulsion, or as a water-in-oil-in-water triple emulsion (such emulsions are known and described, for example, in C. FOX, COSMETICS AND TOILETRIES 101 (Vol. 101.1986).

The cosmetic composition disclosed herein may also comprise cosmetic adjuvants chosen from fatty substances, emollients, softeners, antioxidants, opacifiers, stabilizers, antifoams, moisturizers, vitamins, fragrances, preserving agents, surfactants, fillers, sequestering agents, polymers, acidifying or basifying agents, fragrances, dyes, pigments and thickeners, and any other ingredient used in cosmetics for this type of application.

Needless to say, a person skilled in the art will take care to select this or these optional additional compound(s) such that the advantageous properties intrinsically associated with the cosmetic composition in accordance with the disclosure are not, or are not substantially, adversely affected by the envisaged addition(s).

The surfactants are, for example, chosen from anionic, amphoteric and nonionic surfactants.

The fatty substances may comprise an oil or a wax or a mixture thereof, petroleum jelly, paraffin, lanolin, hydrogenated lanolin or acetylated lanolin; fatty acids; fatty alcohols such as lauryl alcohol, cetyl alcohol, myristyl alcohol, stearyl alcohol, palmityl alcohol, oleyl alcohol and 2-octyldodecanol; fatty acid esters such as glyceryl monostearate, polyethylene glycol monostearate, isopropyl myristate, isopropyl adipate, isopropyl palmitate, octyl palmitate, C₁₂-C₁₅ fatty alkyl benzoates (e.g., Finsolv TN from Finetex), myristyl alcohol polyoxypropylenated with 3 mol of propylene oxide (e.g., Witconol APM from Witco); and C₆-C₁₈ fatty acid triglycerides such as caprylic/capric acid tri-glycerides.

The oils are chosen from animal, plant, mineral and synthetic oils, for example, hydrogenated palm oil, hydrogenated castor oil, liquid petroleum jelly, liquid paraffin, purcellin oil (stearyl octanoate), silicone oils and isoparaffins.

The waxes are chosen from animal, fossil, plant, mineral and synthetic waxes. Mention may be made of beeswaxes, carnauba wax, candelilla wax, sugar cane wax, Japan wax, ozokerites, montan wax, microcrystalline waxes, paraffins and silicone waxes and resins.

The thickeners, which are nonionic, may be chosen from modified and unmodified guar gums and celluloses, such as hydroxypropyl guar gum and cetylhydroxyethylcellulose, silicas, for instance the Bentone Gel MiO sold by the company NL Industries or Veegum Ultra sold by the company Polyplastic.

The cosmetic composition may comprise emollients, which contribute a soft, dry, non-tacky sensation when the composition is applied to the skin. These emollients may be chosen from, for example, volatile silicones, non-volatile silicones and other non-volatile emollients.

Volatile silicones are defined as compounds that are volatile at room temperature. Among these compounds that may be mentioned, for example, are cyclic and linear volatile silicones of the dimethylsiloxane type in which the chains comprise from 3 to 9 silicone residues, such as cyclomethicone D4 or D5.

Non-volatile silicones are defined as compounds with a low vapor pressure at room temperature. The following are examples of these compounds: polyalkylsiloxanes, for example linear polyalkylsiloxanes, such as linear polydimethylsiloxanes or dimethicones, sold by the company Dow Corning under the name “Dow Corning 200 Fluid”; polyalkylarylsiloxanes, such as the polymethylphenylsiloxanes sold by the company Dow Corning under the name “Dow Corning 556 Fluid”; polyether and siloxane copolymers such as dimethicone copolyols.

Examples of the non-volatile emollients that may be used in the present disclosure are: hydrocarbon-based derivatives, mineral oils, fatty alcohols, esters of C₃-C₁₈ alcohols with C₃-C₁₈ acids, esters of benzoic acid with C₁₂-C₁₈ alcohols and mixtures thereof, C₂-C₆ polyols chosen from, for example, glycerol, propylene glycol or sorbitol, and polyalkylene glycol polymers.

The amounts of these various constituents that may be present in the cosmetic composition according to the disclosure are those conventionally used for the presentation forms under consideration.

The cosmetic composition disclosed herein may be in the form of a lotion, a cream or a fluid gel distributed as an aerosol spray, and in this respect may comprise the ingredients generally used in products of this type and well known to those skilled in the art.

The distribution means, which forms a part of the aerosol device, generally comprises at least one distribution valve controlled by a distribution head, which comprises a nozzle via which the aerosol composition is vaporized.

The container containing the pressurized composition may be opaque or transparent.

The container may be made of a material chosen from glass, polymer material or metal, and optionally coated with a layer of protective varnish.

Non-limiting examples illustrating the invention will now be given.

The aerosol device shown below in accordance with the invention was produced by introducing the following fluids and propellants into an aerosol container equipped with standard distribution means:

| Example 1 | Amounts |
|---|---|
| Zinc salicylate trihydrate | 0.99 g |
| Isobutane | 55.0 g |
| Denatured 96° ethyl alcohol | 44.1 g |

The alcohol-based aerosol formulation obtained was transparent and stable after 2 months of storage at 45° C.

Efficacy Test

Underarm sweat was collected from six volunteers in a sauna, the samples of individual sweat, stored in ice for a few hours, are at that point virtually odorless. They were subsequently mixed together and divided into 1 ml aliquots. The active agents were added to these aliquots, which were then incubated in an oven at 37° C. After these samples were incubated for 18 and 24 hours, a panel of experts evaluated the intensity of the odor compared with a control sample, which was 1 ml of sweat incubated without addition of active agent.

The results are expressed below as a percentage of change in the unpleasantness of the odor compared with this control sweat sample (average of the percentages of change at 18 hours and 24 hours).

| | (A) | (B) | (C) |
|---|---|---|---|
| Test amount (mg AM/ml of sweat) | 1 mg | 5 mg | 0.8 mg |
| % change in the unpleasantness of odor | −45% | −48% | −55% |

The active agents (A) and (B) correspond to zinc salicylate trihydrate from Bernardy Chimie and to the active agent (C) containing Triclosan.

Demonstration of the Bactericidal Activity of a Composition With Respect to the Microorganisms Involved in Underarm Odor

The test described herein allows a quantitative determination of the bactericidal activity of a composition on microorganisms under optimum growth conditions, i.e., microorganisms of the Corynebacterium xerosis type (Institut Pasteur Collection No. 5216) grown on soybean tryptocasein agar on a slope.

On the day before the test, 32 g of soybean tryptocasein broth were placed in a pill bottle and incubated at 35° C. On the day of the test, 4 g of the test composition were added and the mixture was homogenized using a Vortex mixer.

A product-free growth control was prepared under the same conditions in order to check that the microorganisms were under favorable growth conditions throughout the duration of the test.

For the preparation of the inoculum, five days before the start of the test, the two bacterial strains were subcultured on a suitable medium. Incubation was performed for 5 days at 35° C. On the day of the test, the slope was washed with about 9 ml of diluent: the suspension obtained has a titre of 108 microorganisms/ml (counting was performed). 4 ml of inoculum were introduced into the pill bottle, which corresponds to a rate of 107 bacteria per gram of preparation. The pill bottle was placed in an incubator-stirrer (35° C.-200 rpm).

After each contact time (2, 4, 6 and 24 hours), the content of the pill bottle was homogenized using a Vortex mixer. Tenfold dilutions were performed. Deposits were placed on the surface of agar Petri dishes (Eugon LT 100 medium). The Petri dishes were incubated for 6 to 7 days in an oven at 35° C.

The colonies were counted on the dishes containing more than 20 and less than 200 colonies.

For the active agent evaluation, the results were expressed, for each test microorganism, as a Log number of reduction after 24 hours relative to the placebo:

| | |
|---|---|
| Identical results: | zero, |
| 1 Log of reduction: | low, |
| 2 Logs of reduction: | moderate, |
| 3 Logs of reduction: | good, |
| ≧4 Logs of reduction: | excellent. |

The following three formulations were prepared:

| | Formula 2 | Formula 3 | Formula 4 |
|---|---|---|---|
| Ingredients | (invention) | (invention) | (prior art) |
| Triclosan | | | 0.1 g |
| Zinc salicylate | 0.1 g | 1 g | |
| Polyethylene glycol | | | 6 g |
| containing 8 EO | | | |
| Acrylic acid/C₁₀—C₃₀ alkyl | | | 0.9 g |
| acrylate crosslinked | | | |
| copolymer | | | |
| Pure sodium hydroxide | | | qs (pH = 7) |
| Microbiologically clean | 99.9 g | 99 g | 93.0 g |
| deionized water | | | |

The bactericidal activity of each of the formulations 2, 3 and 4 with respect to the strain Corynebacterium xerosis was measured and compared with a formulation without active agent.

The results obtained are summarized in the following table:

| | Efficacy at 24 hours relative to the gel |
|---|---|
| Compositions | without active agent |
| Strains | Corynebacterium xerosis |
| 2 (invention) | Low |
| 3 (invention) | Low |
| 4 (prior art) | Excellent |

Zinc salicylate has low antibacterial activity on the Corynebacterium xerosis strains. It thus has a bactericidal activity spectrum that is substantially less broad than that of Triclosan and is friendlier towards the cutaneous flora.

1. A deodorant composition, comprising in a cosmetically acceptable vehicle:
(a) at least one zinc salicylate or a derivative thereof, in free or hydrated form;
(b) at least one C₁-C₄ monoalcohol; and
(c) at least one propellant.

2. The composition according to claim 1, wherein the at least one zinc salicylate or a derivative thereof is chosen from the compounds of formula (I) below: 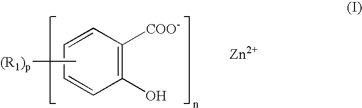
wherein n=2, p is 0, 1, 2 or 3; R₁ is chosen from linear and branched C₁-C₁₈ alkyl radicals; linear and branched C₁-C₁₈ hydroxyalkyl radicals; halogen atoms; C₂-C₁₈ acyl radicals; and radicals chosen from COR₂, OCOR₂ and CONHR₂ wherein R₂ is chosen from hydrogen atoms, and linear and branched C₁-C₁₈ alkyl radicals.

3. The composition according to claim 2, wherein the compound of formula (I) is zinc salicylate (p=0).

4. The composition according to claim 2, wherein the compound of formula (I) is zinc salicylate trihydrate.

5. The composition according to claim 1, wherein the concentration of the at least one zinc salicylate or derivative thereof ranges from 0.001% to 20% by weight relative to the total weight of the composition.

6. The composition according to claim 5, wherein the concentration of the at least one zinc salicylate or derivative thereof ranges from 0.01% to 15% by weight relative to the total weight of the composition.

7. The composition according to claim 6, wherein the concentration of the at least one zinc salicylate or derivative thereof ranges from 0.05% to 5% by weight relative to the total weight of the composition.

8. The composition according to claim 1, wherein the at least one C₁-C₄ mono-alcohol is chosen from methanol, ethanol and isopropanol.

9. The composition according to claim 8, wherein the at least one C₁-C₄ mono-alcohol is ethanol.

10. The composition according to claim 1, wherein the at least one C₁-C₄ mono-alcohol is present at a concentration ranging from 15% to 80% by weight relative to the total weight of the composition.

11. The composition according to claim 10, wherein the at least one C₁-C₄mono-alcohol is present at a concentration ranging from 30% to 70% by weight relative to the total weight of the composition.

12. The composition according to claim 11, wherein the at least one C₁-C₄ mono-alcohol is present at a concentration ranging from 40% to 70% by weight relative to the total weight of the composition.

13. The composition according to claim 1, wherein the at least one propellant is chosen from dimethyl ether, volatile hydrocarbons and volatile fluorohydrocarbons.

14. The composition according to claim 13, wherein the volatile hydrocarbons are chosen from propane, isopropane, n-butane, isobutane, pentane and isopentane, and mixtures thereof.

15. The composition according to claim 13, wherein the volatile fluorohydrocarbons are chosen from 1,1-difluoroethane and 1,1,1-trifluoro-2-fluoroethane, and mixtures thereof.

16. The composition according to claim 1, wherein the at least one propellant is chosen from carbon dioxide, nitrous oxide, nitrogen and compressed air.

17. The composition according to claim 1, wherein the propellant is present at a concentration ranging from 5% to 90% by pressurized weight relative to the total weight of the composition.

18. The composition according to claim 17, wherein the propellant is present in a concentration ranging from 15% to 70% by pressurized weight relative to the total weight of the composition.

19. The composition according to claim 1, wherein the composition further comprises at least one additional active agent chosen from deodorant active agents and antiperspirant active agents.

20. The composition according to claim 19, wherein the additional deodorant active agents are chosen from zinc pyrrolidonecarboxylate; zinc sulphate; zinc chloride; zinc lactate; zinc gluconate; zinc phenolsulphonate; 2,4,4′-trichloro-2′-hydroxydiphenyl ether (Triclosan); 2,4-dichloro-2′-hydroxydiphenyl ether; 3′,4′,5′-trichlorosalicylanilide; 1-(3′,4′-dichlorophenyl)-3-(4′-chlorophenyl)urea (Triclocarban); 3,7,11-trimethyldodeca-2,5,10-trienol (Farnesol), cetyltrimethylammonium salts and cetylpyridinium salts; chlorhexidine and salts thereof; diglyceryl monocaprate; diglyceryl monolaurate and glyceryl monolaurate; and polyhexamethylene biguanide salts.

21. The composition according to claim 19, wherein the antiperspirant active agents are chosen from aluminium chlorohydrate, aluminium chlorohydrex, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, aluminium sulphate, aluminium zirconium octachlorohydrate, aluminium zirconium pentachlorohydrate, aluminium zirconium tetrachlorohydrate, aluminium zirconium trichlorohydrate, and complexes of metals with an amino acid.

22. The composition according to claim 21, wherein the amino acid used with the metal complexes is glycine.

23. The composition according to claim 19, wherein the at least one additional active agent is present in a concentration ranging from 0.001% to 20% by weight relative to the total weight of the composition.

24. The composition according to claim 23, wherein the at least one additional active agent is present in a concentration ranging from 0.1% to 10% by weight relative to the total weight of the composition.

25. The composition according to claim 1, wherein the composition contains no additional deodorant or antiperspirant active agent.

26. The composition according to claim 1, wherein the cosmetically acceptable vehicle further comprises water and/or at least one additional organic solvent.

27. The composition according to claim 1, wherein it is formulated as a water-in-oil or oil-in-water emulsion, or as a water-in-oil-in-water triple emulsion.

28. The composition according to claim 1, wherein the composition further comprises at least one cosmetic adjuvant chosen from fatty substances, emollients, softeners, antioxidants, opacifiers, stabilizers, antifoams, moisturizers, vitamins, fragrances, preserving agents, surfactants, fillers, sequestering agents, polymers, acidifying or basifying agents, fragrances, dyes, pigments and thickeners.

29. The composition according to claim 1, wherein the composition is in the form of a lotion, a cream or a fluid gel.

30. An aerosol device comprising a container comprising an aerosol composition comprising a fluid phase comprising at least one C₁-C₄ monoalcohol, at least one zinc salicylate or a derivative thereof, in free or hydrated form, and at least one propellant; and a means for distributing the aerosol composition.

31. The device according to claim 30, wherein the at least one zinc salicylate or derivative thereof, the at least one monoalcohol and the at least one propellant may be in the same compartment or in different compartments in the aerosol container.

32. A process for treating human body odor comprising applying an aerosol composition to the human body with an aerosol device, wherein the aerosol device comprises a container comprising an aerosol composition comprising a fluid phase comprising at least one C₁-C₄ monoalcohol; at least one zinc salicylate or a derivative thereof, in free or hydrated form; and at least one propellant; and a means for distributing the aerosol composition.

33. The process according to claim 32, wherein the at least one zinc salicylate or derivative thereof, the at least one monoalcohol and the at least one propellant may be in the same compartment or in different compartments in the aerosol container.

34. The process for treating human body odor according to claim 32, wherein the human body odor is underarm odor.
